(19) United States
(12) Patent Application Publication
(10) Pub. No.: US 2026/0248688 A1
(43) Pub. Date: Aug. 27, 2026

(54) IMPROVEMENTS RELATING TO THE STOPPERING OF CONTAINERS

(71) Applicant: BESPAK EUROPE LIMITED, Norfolk (GB)

(72) Inventor: Adam GIBBONS, Norfolk (GB)

(21) Appl. No.: 19/163,576

(22) PCT Filed: Mar. 19, 2024
(86) PCT No.: PCT/EP2024/057231

§ 371 Date: Sep. 9, 2025

(30) Foreign Application Priority Data
Mar. 31, 2023 (GB) 2304784.8

(51) Int. Cl. A61J 1/20; A61J 1/14; A61M 15/08

(52) U.S. Cl. CPC A61J 1/2037; A61J 1/1412; A61M 15/08

(57) ABSTRACT

A dispensing apparatus (100), a container (1) and a valve member (20) are provided wherein the valve member (20) comprises a central body (30) and a flexible skirt (40). The flexible skirt (40) comprises an annular wall (41) having a free edge (42) directed in a first direction. The annular wall (41) defines an annular sealing portion (43) that extends circumferentially around the valve member (20) to provide an unbroken annular sealing surface.

The present disclosure relates to stoppering a container body, containers for fluids and use of such containers in a dispensing apparatus. The container may, for example, be intended for use in a nasal dispensing apparatus.

### BACKGROUND TO THE DISCLOSURE

It is known to provide a metered dose of medicament in a sealed container, such as a vial, to be dispensed from a dispensing apparatus. One example is described in EP 0 827 782 A2 in which a piercing element is provided on a piston that pierces a sealing member in the vial. The sealing member is then displaced into the vial forcing medicament out of the vial through the pierced opening in the sealing member. The medicament then passes through a channel formed in the piston and is expelled from the dispenser. Another example is described in EP 3 820 625 A2 wherein, according to its abstract, a device for dispensing a fluid product comprises a body, a reservoir containing one or two doses of a fluid product, said reservoir comprising a plug which closes off said reservoir in a sealed manner before actuation, a dispensing head provided with a dispensing orifice, and dispensing means for dispensing at least some of said fluid product through said dispensing orifice during an actuation, said device comprising a needle of which the tip is adapted to pierce said plug during the actuation, said reservoir being filled with said fluid product and plugged under vacuum with said plug such that the residual air volume present in said reservoir between said fluid product and said plug is substantially zero.

While sealed containers for fluids such as medicaments are available there remains a need to provide improved, or at least commercially acceptable, alternative containers and means for their stoppering.

### SUMMARY OF THE DISCLOSURE

A first aspect of the present disclosure provides a container for a fluid, the container comprising a container body defining an interior for storage of the fluid and a valve member to close in a storage condition an outlet of the container body;
the valve member comprising a central body and a flexible skirt;
the flexible skirt comprising an annular wall having a free edge directed towards the outlet of the container body, wherein the annular wall defines an annular sealing portion that extends circumferentially around the valve member to provide in the storage condition an unbroken annular seal with the container body;
wherein upon an increase in the pressure of the interior of the container the flexible skirt is configured to deflect inwardly towards the central body to break the annular seal so as to accommodate outflow of fluid through the outlet.

Beneficially, the flexible skirt may be symmetrical about its longitudinal axis. This may beneficially improve the evenness of the shot weight.

The symmetry of the flexible skirt may also beneficially allow for more consistent wall thickness which may allow the valve member to be better aligned within the container body on insertion, i.e. to lessen the risk that the valve member is skewed to one side on insertion.

Beneficially having only a single annular sealing portion, e.g. instead of two axially spaced apart sealing ribs, avoids the risk, when vacuum filling is used, of a negative pressure being trapped between two sealing ribs which can cause a pressure differential out of the container.

Beneficially, the use of the single flexible skirt for sealing may allow the sealing part to be thicker compared to the use of multiple, thinner, sealing ribs. The thicker sealing part may be less liable to permanent deformation when in bulk packaging or when the part is being taken off a mould tool machine. In addition, the thicker sealing part may produce a more reliable, consistent opening under the fluid pressure.

In one embodiment, the annular sealing portion is a portion of the annular wall that is spaced from the free edge of the annular wall.

In one embodiment, the valve member is moulded and comprises a split line of the moulding that is distanced from the annular sealing portion, the split line optionally being located at the free edge of the annular wall. Separating the split line from the annular sealing portion may achieve an improved seal integrity and reliability.

In one embodiment, the annular sealing portion is spaced from the free edge of the annular wall by a distance of 0.5 to 3.0 mm.

In one embodiment, the flexible skirt extends from the central body.

In one embodiment, a second end of the annular wall opposite the free edge is connected with, and optionally integrally formed with, the central body.

In one embodiment, the annular wall is externally convex in an unbiased condition before insertion into the container body.

The annular wall may consist of a smoothly curved wall between the free edge and its second end. In some examples the annular wall has a smooth convexly-curved outer face between its free edge and its second end. In particular, the smooth convexly-curved outer face may be free of raised ribs or other projections. In one embodiment the annular sealing portion consists of a portion of the annular wall that does not project and is not raised relative to a remainder of the annular wall.

The annular wall may be cup-shaped.

In one embodiment, the central body comprises an abutment surface for engaging in use a plunger of a dispensing apparatus for displacing the valve member. The abutment surface may be directed towards the outlet of the container body.

In some embodiments, the central body may comprise a channel for the outflow of fluid, said channel having at least one inlet in fluid communication with the interior of the container when the flexible skirt is deflected. For example, the channel may comprise a transverse conduit formed in a free surface of the abutment surface, and optionally the at least one inlet may be defined at each end of the transverse conduit.

In some other embodiments, the abutment surface may comprise an unbroken annular surface configured to engage a plunger of a dispensing apparatus for displacing the valve member.

In some embodiments, the central body may define a socket for frictionally engaging a plunger of a dispensing apparatus for displacing the valve member.

In one embodiment, the flexible skirt is rotationally symmetric about a longitudinal axis of the valve member passing through a centre of the central body.

The valve member may be displaceable relative to the container body wherein, in use, displacement of the valve member may increase the pressure in the interior of the container.

In one embodiment the valve member is a unitary moulded piece.

The container may be, for example, a vial for medicament.

A second aspect of the present disclosure provides a dispensing apparatus comprising a container according to the first aspect, the dispensing apparatus further comprising a plunger for displacing the valve member relative to the container body.

In one embodiment, the plunger comprises an end part shaped and sized to engage the abutment surface of the central body of the valve member.

In some embodiments, the end part comprises a plug portion for frictionally engaging in the socket of the central body of the valve member.

In some other embodiments, the end part comprises an unbroken annular face for engaging against the central body of the valve member.

In one embodiment, the plunger has a delivery channel for delivering the outflow of fluid from the container.

In some embodiments, the delivery channel may comprise a transverse leg extending inwards from a lateral face of the plunger and a longitudinal leg extending along a longitudinal axis of the plunger.

In some other embodiments, the delivery channel may simply consist of a longitudinal leg extending along a longitudinal axis of the plunger from an end face of the plunger.

In some examples the dispensing apparatus may be a nasal dispensing apparatus. In some examples, the fluid within the container of the dispensing apparatus may consist of a single dose of a nasal medicament.

A third aspect of the present disclosure provides a valve member suitable for sealing an outlet of a container, the valve member comprising a central body and a flexible skirt;
the flexible skirt comprising an annular wall having a free edge directed in a first direction, wherein the annular wall defines an annular sealing portion that extends circumferentially around the valve member to provide an unbroken annular sealing surface. In one embodiment, the annular sealing portion is a portion of the annular wall that is spaced from the free edge of the annular wall. Advantageously this may distance the annular sealing portion from a split line of the valve member. For example the valve member may be moulded and may comprise a split line of the moulding that is distanced from the annular sealing portion, the split line optionally being located at the free edge of the annular wall. Separating the split line from the annular sealing portion may achieve an improved seal integrity and reliability.

In one embodiment the annular sealing portion is spaced from the free edge of the annular wall by a distance of 0.5 to 3.0 mm.

In one embodiment, the flexible skirt extends from the central body.

In one embodiment, a second end of the annular wall opposite the free edge is connected with, and optionally integrally formed with, the central body.

In one embodiment, the annular wall is externally convex in an unbiased condition before insertion into the container body.

In one embodiment, the central body comprises an abutment surface for engaging in use a plunger of a dispensing apparatus for displacing the valve member.

In some embodiments, the central body comprises a channel for the outflow of fluid, said channel having at least one inlet. The channel comprise a transverse conduit formed in a free surface of the abutment surface, and optionally the at least one inlet is defined at each end of the transverse conduit.

In some other embodiments, the abutment surface comprises an unbroken annular surface configured to engage a plunger of a dispensing apparatus for displacing the valve member. In some embodiments, the central body defines a socket for frictionally engaging a plunger of a dispensing apparatus for displacing the valve member.

In one embodiment, the flexible skirt is rotationally symmetric about a longitudinal axis of the valve member passing through a centre of the central body.

In one embodiment, the valve member is a unitary moulded piece.

The container body of the above aspects may be formed from a wide variety of suitable materials, as would be appreciated by a person skilled in the art, such as glass or a plastic polymer. Examples of suitable glass include Type 1 Borosilicate ‘Fiolax Clear’, & Type 2.

Examples of suitable polymers include polypropylene, polyethylene, polystyrene, cyclic olefin copolymer-an example being Topas® COC available from TOPAS Advanced Polymers GmbH, Frankfurt-Höchst, Germany, and liquid crystal polymer—an example being Zenite® LCP available from DuPont, Wilmington, USA.

The container body may consist of an elongate tube. In one embodiment the elongate tube is closed at one open and open at the other end. The elongate tube may be cylindrical. The container body may comprise an outwardly-turned flange at its open end. Alternatively, the rim at the open end may simply consist of an end surface of a tubular element without an outwardly-turned flange.

The valve member of the above aspects may be formed from a wide variety of suitable materials, as would be appreciated by a person skilled in the art, for example an elastomer or thermoelastomer material such as EPDM, polychloroprene, hydrogenated nitrile, butyl, halo-butyl, dynamically cross-linked EPDM/PP (Santoprene®), styrenic block copolymers or blends thereof. Other suitable materials include high-density polyethylene and low-density polyethylene.

The fluid contained within the container of the above aspects may consist of a single dose of fluid or may comprise multiple doses of fluid. The fluid may contain a pharmaceutical.

The term pharmaceutical, as used herein, is intended to encompass any pharmaceutical, compound, composition, medicament, agent or product which can be delivered or administered to a human being or animal, for example pharmaceuticals, drugs, biological and medicinal products. Examples include antiallergics, analgesics, bronchodilators, antihistamines, therapeutic proteins and peptides, antitussives, anginal preparations, antibiotics, anti-inflammatory preparations, hormones, or sulfonamides, such as, for example, a vasoconstrictive amine, an enzyme, an alkaloid, or a steroid, including combinations of two or more thereof. In particular, examples include isoproterenol [alpha-(isopropylaminomethyl) protocatechuyl alcohol], phenylephrine, phenylpropanolamine, glucagon, adrenochrome, trypsin, epinephrine, ephedrine, narcotine, codeine, atropine, heparin, morphine, dihydromorphinone, ergotamine, scopolamine, methapyrilene, cyanocobalamin, terbutaline, rimiterol, salbutamol, ipratropium bromide and salbutamol, flunisolide, colchicine, pirbuterol, beclomethasone, orciprenaline, fentanyl, and diamorphine, streptomycin, penicillin, procaine penicillin, tetracycline, chlorotetracycline and hydroxytetracycline, adrenocorticotropic hormone and adrenocortical hormones, such as cortisone, hydrocortisone, hydrocortisone acetate and prednisolone, insulin, cromolyn sodium, and mometasone, including combinations of two or more thereof. The pharmaceutical may be used as either the free base or as one or more salts conventional in the art, such as, for example, acetate, benzenesulphonate, benzoate, bicarbonate, bitartrate, bromide, calcium edetate, camsylate, carbonate, chloride, citrate, dihydrochloride, edetate, edisylate, estolate, esylate, fumarate, fluceptate, gluconate, glutamate, glycollylarsanilate, hexylresorcinate, hydrobromide, hydrochloride, hydroxynaphthoate, iodide, isethionate, lactate, lactobionate, malate, maleate, mandelate, mesylate, methylbromide, methylnitrate, methylsulphate, mucate, napsylate, nitrate, pamoate, (embonate), pantothenate, phosphate, diphosphate, polygalacturonate, salicylate, stearate, subacetate, succinate, sulphate, tannate, tartrate, and triethiodide, including combinations of two or more thereof. Cationic salts may also be used, for example the alkali metals, e.g. Na and K, and ammonium salts and salts of amines known in the art to be pharmaceutically acceptable, for example glycine, ethylene diamine, choline, diethanolamine, triethanolamine, octadecylamine, diethylamine, triethylamine, 1-amino-2-propanol-amino-2-(hydroxymethyl)propane-1,3-diol, and 1-(3,4-dihydroxyphenyl)-2 isopropylaminoethanol. The pharmaceutical will typically be one which is suitable for nasal inhalation and may be provided in any suitable fluid form for this purpose, for example as a solution or powder suspension in a solvent or carrier liquid, for example ethanol, or isopropyl alcohol. The pharmaceutical may alternatively be one suitable for sub-lingual or ophthalmic delivery. The pharmaceutical may, for example, be one which is suitable for the treatment of asthma. Examples include salbutamol, beclomethasone, salmeterol, fluticasone, formoterol, terbutaline, sodium chromoglycate, budesonide and flunisolide, and physiologically acceptable salts (for example salbutamol sulphate, salmeterol xinafoate, fluticasone propionate, beclomethasone dipropionate, and terbutaline sulphate), solvates and esters, including combinations of two or more thereof. Individual isomers such as, for example, R-salbutamol, may also be used. The pharmaceutical may, for example, be one which is suitable for the treatment of migraine. An example is sumatriptan. As will be appreciated, the pharmaceutical may comprise of one or more active ingredients, an example of which is flutiform. One or more surfactants may be included if desired.

### BRIEF DESCRIPTION OF THE DRAWINGS

One or more embodiments of the disclosure will now be described, by way of example only, with reference to the accompanying drawings in which:

FIG. 1 is a schematic illustration of a container containing a fluid;

FIG. 2 is a perspective view of a first embodiment of valve member of the container of FIG. 1;

FIG. 3 is a cross-sectional view of the valve member of FIG. 2;

FIG. 4 is a schematic illustration showing the interaction of a second embodiment of valve member and a plunger of a dispensing apparatus;

FIG. 5 is a schematic illustration showing the interaction of a third embodiment of valve member and a plunger of a dispensing apparatus; and

FIG. 6 is a perspective view of the third embodiment of valve member.

### DETAILED DESCRIPTION

Unless defined otherwise, all technical and scientific terms used in this specification have the same meaning as is commonly understood by the reader skilled in the art to which the claimed subject matter belongs. It is to be understood that the foregoing summary of the disclosure and the following examples are exemplary and explanatory only and are not restrictive of any subject matter claimed.

The following description is directed to embodiments of the disclosure. The description of the embodiments is not meant to include all the possible embodiments of the disclosure that are claimed in the appended claims. Many modifications, improvements and equivalents which are not explicitly recited in the following embodiments may fall within the scope of the appended claims. Features described as part of one embodiment may be combined with features of one or more other embodiments unless the context clearly requires otherwise.

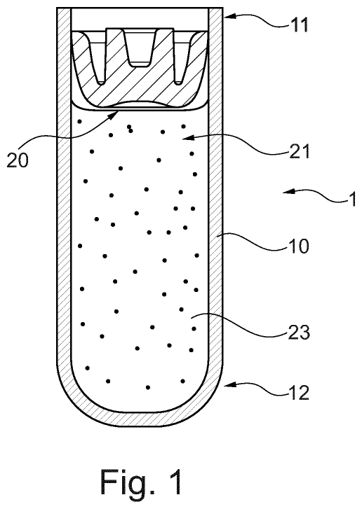

FIG. 1 shows a container 1 containing a fluid 23. The container 1 comprises a container body 10 that is open at a first end 11 and closed at a second end 12.

The container 1 further comprises a valve member 20 that is inserted into the container body 10 via its open end 11. The container body 10 defines an interior 21 of the container 1 that is sealed in a storage condition by the valve member 20.

The container body 10 once sealed using valve member 20 forms the container 1. In one embodiment the container 1 consists only of the two components of the container body 10 and the valve member 20.

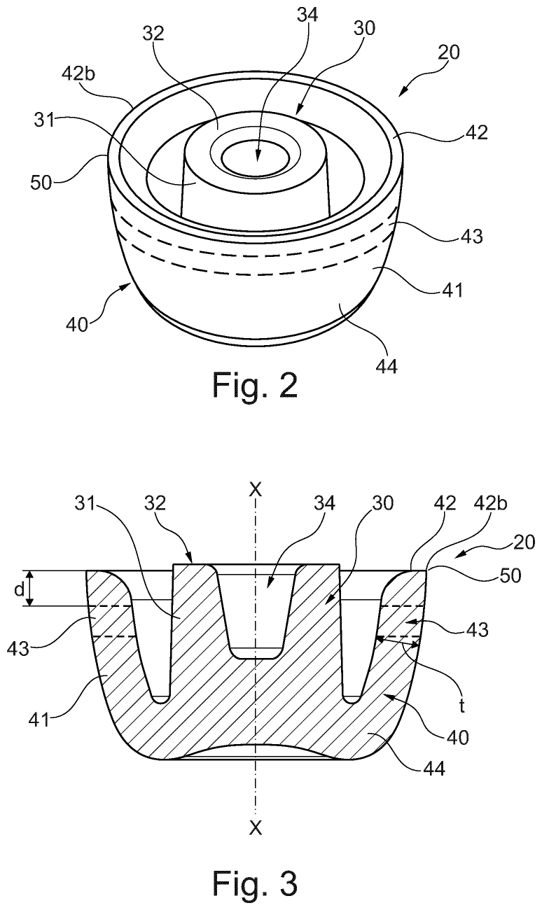

FIGS. 2 and 3 show a first embodiment of the valve member 20 in more detail. The valve member 20 is suitable for sealing an outlet of the container 1, for example the outlet formed by the open end 11. The valve member 20 comprises a central body 30 and a flexible skirt 40. The flexible skirt 40 comprises an annular wall 41 having a first edge that is a free edge 42 directed in a first direction, which in the illustrated example of FIG. 1 is towards the open end 11 of the container 1.

The valve member 20 may be a unitary moulded piece. The valve member 20 may comprise a split line 50 arising from the mould tools that may be used to form the valve member 20. The split line 50 may be located at the free edge 42, for example at an outer corner 42b of the free edge 42.

The flexible skirt 40 is in one embodiment rotationally symmetric about a longitudinal axis X-X of the valve member 20 passing through a centre of the central body 30.

The annular wall 41 defines an annular sealing portion 43 that extends circumferentially around the valve member 20 to provide an unbroken annular sealing surface in use. The annular sealing portion 43 is illustrated, by way of example, in FIGS. 2 and 3 as the region of the annular wall 41 between the broken lines.

The annular sealing portion 43 is in one embodiment a portion of the annular wall 41 that is spaced from the free edge 42 of the annular wall 41. The annular sealing portion 43 may be spaced from the free edge 42 by a distance, d, as shown in FIG. 3. Distance, d, may be, for example in the range of 0.5 to 3.0 mm. The annular sealing portion 43 may thus also be distanced from the split line 50 of the valve member 20.

The flexible skirt 40 may extend from the central body 30. In one embodiment, a second end 44 of the annular wall 41 opposite the first, free edge 42 is connected with, and in one embodiment integrally formed with, the central body 30.

The annular wall 41 may be externally convex in an unbiased condition before insertion into the container body 10 as shown in FIG. 3.

The central body 30 may comprise an element 31 that projects in a first direction from, for example, the second end 44 of the flexible skirt 40. The element 31 may comprise or consist, for example, of a cylindrical or frusto-conical element. The element 31 may be solid but is in one embodiment hollow along at least a part of its length.

The central body 30 may comprise an abutment surface 32 for engaging in use a plunger of a dispensing apparatus for displacing the valve member 20. The abutment surface 32 may be a distal, free surface of the element 31. The abutment surface 32 my lie in a plane that is perpendicular to the longitudinal axis X-X of the valve member 20.

In the embodiment of FIGS. 2 and 3, the abutment surface 32 comprises an unbroken annular surface configured to engage a plunger.

The central body 30, for example the element 31, may optionally define a socket 34 for frictionally engaging the plunger of the dispensing apparatus.

In one embodiment, in an unbiased condition (before insertion into the container body 10), the flexible skirt 40 of the valve member 20 has a largest external diameter that is greater than an internal diameter of the container body 10. The largest external diameter of the flexible skirt 40 may be the external diameter of the free edge 42, in particular the external diameter of the outer corner 42b. In some examples the largest external diameter may be 5.50 to 6.00 mm, optionally 5.50 mm. For such examples the internal diameter of the container body 10 may be 4.50 to 5.20 mm, optionally 4.90 to 5.10 mm.

The flexible skirt 40 may have an overall height (measured parallel to the longitudinal axis X-X) of 2.50 to 5.00 mm, optionally 3.50 mm. The thickness, t, of the flexible skirt 40 may be 0.60 to 0.80 mm, optionally 0.70 mm. The flexible skirt 40 may be tapered at and/or towards the free edge 42.

The container 1 may be filled and sealed by first filling the fluid 23, via the open first end 11, into the container body 10 until the desired volume is present. Thereafter, the valve member 20 may be inserted into the container body 10, again via the open first end 11 and displaced down the container body 10 until it is proximate or in contact with the fluid 23. In some examples, a headspace between the valve member 20 and the fluid 23 may be 0.5 to 3.00 mm. Upon inserting the valve member 20 into the container body 10 the flexible skirt 40 is flexed inwards such that the annular sealing portion 43 is engaged against the inner face of the container body 10. Optionally, this flexing of the flexible skirt 40 may also move the outer corner 42b of the free edge 42 out of contact with the inner face of the container body 10 so as to move the split line 50 out of contact with the inner face of the container body 10.

The filling and sealing steps may be carried out if desired under a vacuum or partial vacuum. If a substantial amount of gas is present in a headspace of the container body 11 (for example where no vacuum or a low level of partial vacuum is applied) during sealing with the valve member 20, the downward movement of the valve member 20 may be accompanied by inward flexing of the flexible skirt 40 to temporarily break the seal between the annular sealing portion 43 and the container body 10 to allow by-pass of gas from the decreasing volume of the headspace around the valve member 20 as it is moved down the container body 10.

In use, the container 1 may be dispensed using a variety of dispensing apparatus. Typically, common to the dispensing apparatus will be the provision of a plunger for displacing the valve member 20 relative to the container body 10.

For the embodiment of valve member 20 shown in FIGS. 2 and 3, the plunger will typically comprise an end part shaped and sized to engage the abutment surface 32 of the central body 30 of the valve member 20 and/or having a plug portion sized and shaped to engage in the socket 34 of the element 31.

Dispensation of fluid 23 is achieved by using the plunger to displace the valve member 23 further towards the second end 12 of the container body 10. Doing so pressurises the fluid 23 to a point where the sealing force between the annular sealing portion 43 and the inner face of the container body 10 is overcome to break the seal and the fluid 23 passes between the flexible skirt 40 and the inner face of the container body 10.

The subsequent flow path of the fluid 23 is determined by the particular configuration of the valve member 20 and the plunger.

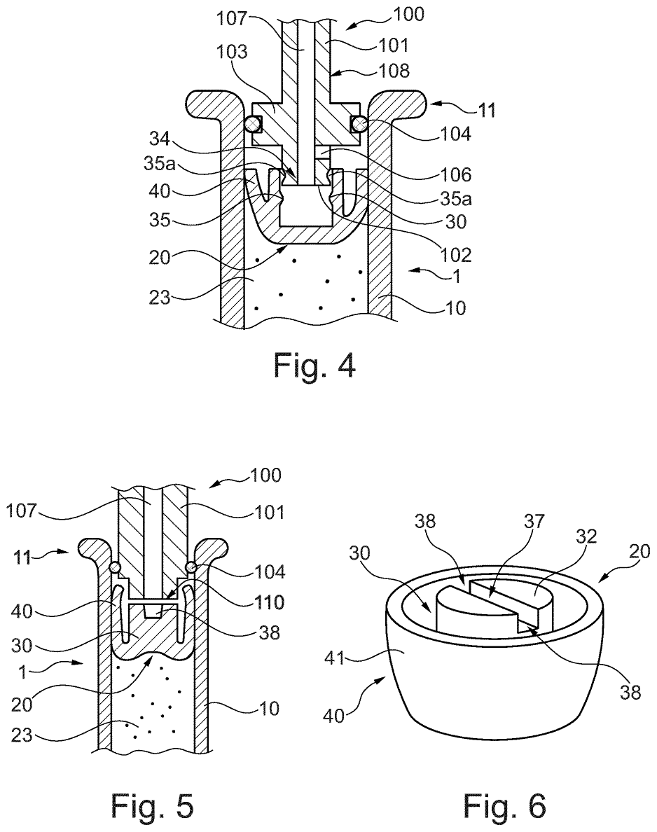

For the valve member 20 of FIGS. 2 and 3 the plunger will typically define the flow path. For example the plunger may have a delivery channel for delivering the outflow of fluid 23 from the container 1. FIG. 4 shows one example of a suitable plunger 101 of a dispensing apparatus 100. FIG. 4 is shown with a second embodiment of the valve member 20 but could also be used or adapted for use with the first embodiment of valve member 20. The second embodiment of the valve member 20 is substantially the same as the first embodiment shown in FIGS. 2 and 3 except that the socket 34 is shaped to have a more cylindrical form and comprises a retaining rib 35 on its inner face.

In the example of FIG. 4, the plunger 101 comprises a plug portion 102. The plug portion 102 is provided with an annular recess 35a on its outer face. The plug portion 102 is sized to be engaged in the socket 34 whereupon the retaining rib 35 is frictionally with the complementary annular recess 35a. Alternatively, the plug portion 102 may not have a recess and the socket 34 may not have a retaining rib such that a solely frictional fit between the plug portion 102 and the valve member 20 is utilised.

The plunger 101 may further comprise a flange 103 having an annular seal 104. Further, the delivery channel 105 of the plunger may comprise a transverse leg 106 extending inwards from a lateral face 108 of the plunger 101 (optionally through a side wall of the plug portion 102) and a longitudinal leg 107 extending along a longitudinal axis of the plunger 101.

The plunger 101 of FIG. 4 is suitable for use with valve members 20 (such as those shown in FIGS. 2 to 4) where the abutment face 32 is an unbroken annular face, since the plunger 101 provides the transverse leg of the delivery channel.

In use the plunger 101 is used to displace the valve member 20 as described above. In the example of FIG. 4, the force from the plunger 101 may be applied to the central body 30 via the plug portion 102 and/or the flange 103 to move the valve member 20 within the container body 10. As noted above, doing so pressurises the fluid 23 to the point where the sealing force between the annular sealing portion 43 and the inner face of the container body 10 is overcome and the fluid 23 passes between the flexible skirt 40 and the inner face of the container body 10. The pressurised fluid 23 that has passed the valve member 20 is channelled along the transverse leg 106 and then along the longitudinal leg 107 upwards to a dispensing nozzle or orifice of the dispensing apparatus (not shown). Escape of the fluid 23 between the flange 103 of the plunger and the container body 10 is prevented by action of the annular seal 104.

The plug portion 102 of the plunger 101 may be removed if not desired such that an end face of the plunger 101 simply engages in face-to-face contact with the abutment face 32 of the central body 30.

FIGS. 5 and 6 show a third embodiment of the valve member 20. The third embodiment of the valve member 20 is substantially the same as the first embodiment shown in FIGS. 2 and 3 except that the central body 30 comprises a channel 37 for the outflow of the fluid 23, said channel 37 having at least one inlet 38. In the illustrated example the channel 37 comprises a transverse conduit formed in the free surface of the abutment surface 32, and two inlets 38 are defined, one at each end of the transverse conduit.

The plunger 101 of the dispensing apparatus 100 has an end part that comprises an unbroken annular face 110 for engaging against the central body 30 of the valve member 20. Thus, in the example of FIG. 5 the delivery channel of the plunger 101 comprises only a longitudinal leg 107 and the fluid 23 reaches the longitudinal leg 107 via the transverse conduit of the valve member 20 rather than a transverse leg provided in the plunger 101. In other respects the dispensing apparatus 100 operates in the same manner as described above.

The dispensing apparatus 100 is provided merely as an example and it will be understood that the embodiments of container 1 may be used with other apparatus configured to dispense the fluid 23.

It is to be understood that at least some of the figures and descriptions of the disclosure have been simplified to focus on elements that are relevant for a clear understanding of the disclosure, while eliminating, for purposes of clarity, other elements that the reader skilled in the art will appreciate may also be present. Because such elements are well known to the reader skilled in the art, and because they do not necessarily facilitate a better understanding of the disclosure, a description of such elements is not provided herein.

1. A container for a fluid, the container comprising a container body defining an interior for storage of the fluid and a valve member to close in a storage condition an outlet of the container body;
the valve member comprising a central body and a flexible skirt;
the flexible skirt comprising an annular wall having a free edge directed towards the outlet of the container body, wherein the annular wall defines an annular sealing portion that extends circumferentially around the valve member to provide in the storage condition an unbroken annular seal with the container body;
wherein upon an increase in the pressure of the interior of the container the flexible skirt is configured to deflect inwardly towards the central body to break the annular seal so as to accommodate outflow of fluid through the outlet.

2. The container of claim 1, wherein the annular sealing portion is a portion of the annular wall that is spaced from the free edge of the annular wall.

3. The container of claim 1, wherein the valve member is moulded and comprises a split line of the moulding that is distanced from the annular sealing portion.

4. (canceled)

5. The container of claim 1, wherein the flexible skirt extends from the central body;
and optionally wherein a second end of the annular wall opposite the free edge is connected with, and optionally integrally formed with, the central body.

6. The container of claim 1, wherein the annular wall is externally convex in an unbiased condition before insertion into the container body.

7. The container of claim 1, wherein the central body comprises an abutment surface for engaging in use a plunger of a dispensing apparatus for displacing the valve member;
and optionally wherein the abutment surface is directed towards the outlet of the container body.

8. The container of claim 7, wherein the central body comprises a channel for the outflow of fluid, said channel having at least one inlet in fluid communication with the interior of the container when the flexible skirt is deflected;
and optionally wherein the channel comprises a transverse conduit formed in a free surface of the abutment surface, and optionally the at least one inlet is defined at each end of the transverse conduit.

9. The container of claim 7, wherein the abutment surface comprises an unbroken annular surface configured to engage a plunger of a dispensing apparatus for displacing the valve member.

10. The container of claim 1, wherein the central body defines a socket for frictionally engaging a plunger of a dispensing apparatus for displacing the valve member.

11. (canceled)

12. (canceled)

13. The container of claim 1, wherein the valve member is a unitary moulded piece.

14. (canceled)

15. A dispensing apparatus comprising a container according to claim 1, the dispensing apparatus further comprising a plunger for displacing the valve member relative to the container body.

16. The dispensing apparatus of claim 15, wherein the plunger comprises an end part shaped and sized to engage the abutment surface of the central body of the valve member.

17. The dispensing apparatus of claim 16, wherein the end part comprises a plug portion for frictionally engaging in the socket of the central body of the valve member.

18. The dispensing apparatus of claim 16, wherein the end part comprises an unbroken annular face for engaging against the central body of the valve member.

19. The dispensing apparatus of claim 15, wherein the plunger has a delivery channel for delivering the outflow of fluid from the container; and optionally wherein the delivery channel comprises a transverse leg extending inwards from a lateral face of the plunger and a longitudinal leg extending along a longitudinal axis of the plunger.

20. A valve member suitable for sealing an outlet of a container, the valve member comprising a central body and a flexible skirt;
the flexible skirt comprising an annular wall having a free edge directed in a first direction, wherein the annular wall defines an annular sealing portion that extends circumferentially around the valve member to provide an unbroken annular sealing surface.

21. The valve member of claim 20, wherein the annular sealing portion is a portion of the annular wall that is spaced from the free edge of the annular wall.

22. The valve member of claim 20, wherein the valve member is moulded and comprises a split line of the moulding that is distanced from the annular sealing portion, the split line optionally being located at the free edge of the annular wall.

23. (canceled)

24. The valve member of claim 20, wherein the annular wall is externally convex in an unbiased condition before insertion into the container body.

25. The valve member of claim 20, wherein the central body comprises an abutment surface for engaging in use a plunger of a dispensing apparatus for displaceing the valve member.
